SEQUENCE LISTING (7 sequences)

SEQ ID NO: 1 - DNA, 1092 bp, Rattus rattus
  CDS (82)...(555)
aggggaagtcccgagacaaaggagcgccgccgcctctgccgccgcgacggtccgggccgcggtcgcccagggactttgaatatgtcggggattgccctcagccgacttgcgcaggagaggaaagcctggaggaaggaccacccttttggctttgtggctgtcccaacaaagaaccctgatggcacgatgaacctgatgaactgggagtgtgctatccctggaaagaaggggactccgtgggaaggaggcttgttcaagctacggatgcttttcaaagatgactatccgtcctcaccaccaaaatgtaaatttgagccaccactgtttcatccaaacgtgtatccttctggcacagtgtgcctgtccatcctggaggaagacaaggactggcggccagctattacgatcaaacagatcttattaggaatacaagaacttctaaatgaaccaaatattcaagacccagctcaagcagaggcctatacaatttactgccaaaacagagtggaatatgagaaaagggttcgagcacaagcgaagaagtttgccccctcataagcagcggcccgggctccatgacgaggaagggattggcttggcaagaacttgtttacaaccttttgcagatctaagtcgctccgtacagttactagtagcctgggagggttgagcgggcgccattttccatttccgccactggcatattcagtcttttgtatttttgattattgagtaaaacttgcttttattttaatattgatgtcagtatttcaactgctgtaaaatgataaacttttgtacttggtaagccctaggagctagtttcttctcgtccgctcggatcgaggcatgttccccactgttcagagctctggcctccagctggctgtatgacagaaccacactgtccctccttccttccctaccctcgtccttctcagaaacctgggctgttgcttatgagcctcagatccaaagttggccagcatctccattctgcaccacttcctttgtgtttatatggcgttttgtctgtgttgctgtttagagtaaataaactgtttatataaaaaaaaaaaaaaaaaaaaaaaa

SEQ ID NO: 2 - protein, 158 aa, Rattus rattus [sequence omitted]

SEQ ID NO: 3 - DNA, 24 bp, Mus musculus
acggtatctgatcgtcttcgaacc

SEQ ID NO: 4 - protein, 16 aa, Artificial Sequence
  Synthetically generated peptide
MASYPYDVPDYASPEF

SEQ ID NO: 5 - protein, 157 aa, Artificial Sequence [sequence omitted]

SEQ ID NO: 6 - DNA, 1080 bp, Saccharomyces cerevisiae
ttttttttttttatataaacagtttatttactctaaacagcaacacagacaaaacgccatataaacacaaaggaagtggtgcagaatggagatgctggccaactttggatctgaggctcataagcaacagcccaggtttctgagaaggacgagggtagggaaggaaggagggacagtgtggttctgtcatacagccagctggaggccagagctctgaacagtggggaacatgcctcgatccgagcggacgagaagaaactagctcctagggcttaccaagtacaaaagtttatcattttacagcagttgaaatactgacatcaatattaaaataaaagcaagttttactcaataatcaaaaatacaaaagactgaatatgccagtggcggaaatggaaaatggcgcccgctcaaccctcccaggctactagtaactgtacggagcgacttagatctgcaaaaggttgtaaacaagttcttgccaagccaatcccttcctcgtcatggagcccgggccgctgcttatgagggggcaaacttcttcgcttgtgctcgaacccttttctcatattccactctgttttggcagtaaattgtataggcctctgcttgagctgggtcttgaatatttggttcatttagaagttcttgtattcctaataagatctgtttgatcgtaatagctggccgccagtccttgtcttcctccaggatggacaggcacactgtgccagaaggatacacgtttggatgaaacagtggtggctcaaatttacattttggtggtgaggacggatagtcatctttgaaaagcatccgtagcttgaacaagcctccttcccacggagtccccttctttccagggatagcacactcccagttcatcaggttcatcgtgccatcagggttctttgttgggacagccacaaagccaaaagggtggtccttcctccaggctttcctctcctgcgcaagtcggctgagggcaatccccgacatattcaaagtccctgggcgaccgcggcccggaccgtcgcggcggcagaggcggcggcgctcctttgtctcgggacttcccct

SEQ ID NO: 7 - protein, 157 aa, Artificial sequence
  Consensus sequence
MSXXXLXRLXXERKXWRKDHPFGFXAXPXKXXDGXMXLXXWEXXIPGKXGTXWXGGXXXXXXXXXXXYPSXPPKXKFXXXXXHPNVYPSGTXCLSILXEDXDWRPAITXKQIXLGXQXLLXXPNXXXPAQXXAXXXXXXNXXEYXKXVXXQAKXXXX

(19) United States
(12) Patent Application Publication
(10) Pub. No.: US 2002/0128189 A1
(43) Pub. Date: Sep. 12, 2002

(54) Ubiquitination of the transcription factor E2A

(75) Inventors: Choon-Joo Kho, Singapore (SG); Mu-En Lee, Newton, MA (US); Edgar Haber, (US); Carol Haber, Salisbury, NH (US)

Correspondence Address: JANIS K. FRASER, PH.D., J.D., Fish & Richardson P.C., 225 Franklin Street, Boston, MA, 02110-2804, US

(73) Assignee: President and Fellows of Harvard College, a Massachusetts corporation

(21) Appl. No.: 09/785,671

(22) Filed: Feb. 16, 2001

Related U.S. Application Data
(63) Continuation of application No. 08/986,605, filed on Dec. 8, 1997, now abandoned.
Application No. 08/986,605 is a continuation of application No. 08/825,476, filed on Mar. 28, 1997, now abandoned.
(60) Provisional application No. 60/014,388, filed on Mar. 28, 1996.

(51) Int. Cl. A61K 48/00; A61K 38/17

(52) U.S. Cl. 514/12; 514/44

(57) ABSTRACT

Disclosed is a polypeptide termed UBCE2A that catalyzes the covalent attachment of ubiquitin to the transcription factor E2A, thereby triggering the degradation of E2A. Also disclosed are DNAs encoding UBCE2A.

This application claims benefit from U.S. Ser. No. 60/014,388, filed Mar. 28, 1996, and U.S. Ser. No. 08/825,476, filed Mar. 28, 1997.

The field of the invention is regulation of transcription factors.

### BACKGROUND OF THE INVENTION

The E2A gene encodes two proteins, E12 and E47, through alternative splicing using two adjacent basic helix-loop-helix (bHLH) coding exons (Sun et al., 1991, Cell 64:459-470). These proteins belong to a family of eukaryotic transcription factors that contain a highly conserved HLH motif, which mediates dimerization, and an adjacent basic region, which is responsible for site-specific DNA binding (Murre et al., 1989, Cell 56:777-783; Murre et al., 1989, Cell 58:537-544). E12 and E47 were initially identified in B cells as immunoglobulin enhancer-binding proteins but were subsequently found to be widely expressed (Roberts et al., 1993, Proc. Natl. Acad. Sci. USA 90:7583-7587).

The E2A proteins are capable of forming heterodimers with tissue-specific HLH proteins, which then bind to DNA and upregulate the transcription of target genes. Tissue-specific HLH proteins include the MyoD family, which is involved in skeletal muscle differentiation (Weintraub, 1993, Cell 75:1241-1244); the achaete-scute family, which is involved in neuronal differentiation (Guillemot et al., 1993, Cell 75:463-476); and the SCL/TAL gene, which is involved in hematopoiesis (Hsu et al., 1991, Mol. Cell. Biol. 11:3037-3042). E2A proteins can also form homodimers and it has been shown that an intermolecular disulfide bond cross-links E2A homodimers in B cells but not in muscle cells (Benezra, 1994, Cell 79:1057-1067). Homodimers are thought to be the predominant DNA-binding species in B cells (Murre et al., 1991, Mol. Cell. Biol. 11:1156-1160). Mice carrying a null mutation in E2A failed to rearrange their immunoglobulin gene segments and lack mature B lymphocytes (Bain et al., 1994, Cell 79:885-892; Zhuang et al., 1994, Cell 79:875-884).

The E2A gene has also been found to be the breakpoint of two translocations associated with childhood lymphoid leukemia. The E2A gene is truncated and fused to either the PBX1 homeobox gene (Kamps et al., 1990, Cell 60:547-555; Nourse et al., 1990, Cell 60:535-545) or the HLF basic leucine zipper gene (Yoshihara et al., 1995, Mol. Cell. Biol. 15:3247-3255). In both instances, the E2A portion is required for transformation.

### SUMMARY OF THE INVENTION

The present invention is based upon the discovery of a natural cellular mechanism for regulating the level of the transcription factor E2A (E12/E47) within a cell. This mechanism relies upon a novel nuclear ubiquitin-conjugating enzyme, termed UBCE2A, which binds to and ubiquitinates E2A, thus targeting it for destruction by the ubiquitin-proteasome pathway. Furthermore, it has been shown that downregulation of E2A by the ubiquitin-proteasome pathway is required for cell cycle progression. Therefore, cellular proliferation in vivo can be regulated by modulating the UBCE2A-mediated degradation of E2A.

The term UBCE2A is herein defined as encompassing a protein, the sequence of which is identical to SEQ ID NO.:2, as well as all naturally occurring splice variants and mammalian homologues capable of ubiquitinating mammalian E2A. The invention features a substantially pure polypeptide that regulates the level of E2A within a cell by catalyzing the covalent attachment of ubiquitin to E2A. This polypeptide may be encoded by a naturally-occurring mRNA transcript, e.g., a transcript approximately 1.1, 1.5, or 2.1 kb long. Preferably, the polypeptide is at least 70%, more preferably at least 80% (e.g., at least 85% or even 90%), and most preferably at least 95% identical to rat UBCE2A (SEQ ID NO.:2) when analyzed by standard means, using the Sequence Analysis Software Package developed by the Genetics Computer Group (University of Wisconsin Biotechnology Center, Madison, Wis.), or an equivalent program (see e.g., Ausubel et al., 1993, Current Protocols in Molecular Biology, New York: John Wiley and Sons), employing the default parameters thereof. In the case of amino acid sequences that are less than 100% identical to a reference sequence, the non-identical positions are preferably, but not necessarily, conservative substitutions for the equivalent positions in the reference sequence. However, whether or not a substitution is conservative does not affect the percent sequence identity, which registers only identity or non-identity. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. The polypeptide of the invention can have the sequence of a naturally occurring protein, e.g., a mammalian UBCE2A such as a human, rat, mouse, guinea pig, hamster, rabbit, dog, cat, cow, horse, pig, goat, sheep, monkey, or ape protein. Alternatively, it may differ from a naturally occurring protein by deletion, addition, or substitution of one or more amino acid residues. In particular, from one to all of the 29 carboxy-terminal residues of rat UBCE2A (SEQ ID NO.:2), or the corresponding residues of any mammalian UBCE2A, may be deleted or replaced by different residues. In addition, the polypeptide may be recombinantly fused to a second polypeptide (e.g., a signal sequence or antigenic sequence) to form a useful chimera that is secreted or readily purified, respectively. The polypeptide may be purified from a biological sample, chemically synthesized, or produced recombinantly. For example, a polypeptide of the invention may be obtained by culturing cells that express the polypeptide and harvesting it from the cells or from the medium surrounding the cells. The invention also features substantially pure polypeptides that consist of mutant forms of the mammalian transcription factor E2A. The mutants may differ form E2A, for example, by being unable to bind UBCE2A or by lacking one or more of the lysine residues that are ubiquitination sites on wild type E2A.

Once purified, the recombinant polypeptide may be used to generate antibodies that specifically bind UBCE2A. These antibodies may be prepared by a variety of standard techniques. For example, the UBCE2A polypeptide, or an antigenic fragment thereof, can be administered to an animal in order to induce the production of polyclonal antibodies. Alternatively, standard hybridoma technology can be used to prepare monoclonal antibodies. In addition, genetically engineered, neutralizing, or humanized antibodies that bind UBCE2A can be generated by well known methods, as can antibody fragments, including F(ab′)2, Fab′, Fab, Fv, and sFv fragments.

The invention also features isolated DNA molecules, including (1) single- or double-stranded molecules encoding the UBCE2A-related polypeptides described above, including polypeptides that have the sequence of rat UBCE2A (SEQ ID NO.:2) or that differ from this sequence by deletion, addition, or substitution of one or more amino acid residues; (2) single-stranded molecules that are antisense to at least a portion of the coding strand of a naturally-occurring gene encoding UBCE2A or to UBCE2A mRNA; and (3) single- or double-stranded molecules having a strand that hybridizes to a probe consisting of a sequence complementary to the coding sequence of UBCE2A (SEQ ID NO.:1) when hybridized and washed under the following stringency conditions: 55° C., 0.1×SSC, 0.1% SDS. The DNA may be transcribed into an mRNA that is approximately 1.1, 1.5, or 2.1 kilobases in length. The DNA or its corresponding RNA may be incorporated into a vector, such as a plasmid, adenovirus, or retrovirus, using standard recombinant techniques. These vectors will have numerous uses. For example, they will have therapeutic applications, as discussed below, and they will be useful for transfecting or transforming cells, thus providing a way to obtain large amounts of the polypeptide of the invention. Indeed, another feature of the invention is a cell that contains a vector encoding a polypeptide that ubiquitinates E2A.

A human patient who is suffering from an undesirable growth of cells could benefit from receiving a treatment that prevents, or at least decreases, the ubiquitination, and subsequent degradation, of E2A. For all methods of treatment, a patient is first identified as having a cell or a class of cells, the proliferation of which is susceptible to inhibition when the level of E2A within the cell is increased. The treatment may involve administering a compound that reduces the level of UBCE2A biological activity. This could be accomplished, for example, by administering an anti-UBCE2A antibody; or a single-stranded nucleic acid molecule that is antisense to at least a portion of the coding strand of a naturally-occurring gene or mRNA encoding UBCE2A; or a peptide having the sequence of a portion or all of (a) the E2A binding site on UBCE2A, or (b) the UBCE2A binding site on E2A. Alternatively, E2A degradation may be inhibited by introducing proteasome inhibitors into the cell. Yet another therapeutic intervention would be administration of a mutant form of E2A that possesses the DNA-binding and transcription factor activities of wild type E2A, but that cannot be ubiquitinated by UBCE2A. This could be accomplished by genetic therapy, targeting the cells of interest, or by administering the genetically engineered polypeptide itself. These treatment regimes are discussed more fully below.

By “polypeptide” is meant any chain of more than two amino acids, regardless of post-translational modifications such as glycosylation or phosphorylation.

By “substantially pure polypeptide” is meant any polypeptide that is substantially free from the components that naturally accompany it. Typically, a polypeptide is substantially pure when at least 60%, preferably at least 75%, more preferably at least 90%, and most preferably at least 99% by weight of the total material in a sample is the polypeptide of interest. Purity can be measured by any appropriate method, e.g., by column chromatography, polyacrylamide gel electrophoresis, or HPLC analysis. A recombinant polypeptide produced in a heterologous expression system is by definition “substantially pure” when made, since it is in a milieu which differs from its natural milieu.

By “isolated DNA” is meant a single- or double-stranded DNA that is not immediately contiguous with, i.e. covalently linked to, either of the coding sequences with which it is immediately contiguous in the naturally occurring genome of the organism from which the DNA of the invention was originally derived. The term therefore includes, for example: a recombinant DNA that is incorporated into a vector, such as an autonomously replicating virus or plasmid; a recombinant DNA that is incorporated into the genomic DNA of a prokaryote or eukaryote at a site different than its original site in its original genome; a recombinant DNA that is part of a hybrid gene encoding additional polypeptide sequence(s); and DNA that exists as a separate molecule independent of other DNA sequences, for example a cDNA or genomic DNA fragment produced by a biochemical reaction, such as the polymerase chain reaction (PCR), ligase chain reaction, or restriction endonuclease treatment. Also included in the isolated DNAs of the invention are single-stranded DNAs that are generally at least 8 nucleotides long, preferably at least 12 nucleotides long, more preferably at least 30 (e.g., at least 50 or 100) nucleotides long, and ranging up to the full-length of the gene or cDNA encoding an UBCE2A polypeptide. The single-stranded DNAs can be detectably labelled for use as hybridization probes, and can be sense or antisense.

By “an antibody that specifically binds” to a given protein is meant an antibody that binds to that protein and that does not substantially recognize and bind to other unrelated molecules. By “neutralizing antibody” is meant an antibody that interferes with the biological activity of UBCE2A. The biological activity described herein is the ubiquitination of E12. The neutralizing antibody may reduce or prevent the degradation of E12.

By “proteasome inhibitor” is meant any compound that inhibits the proteolytic activity of the proteasome. Encompassed by this definition are peptide-aldehydes that include but are not limited to inhibitors of the 20S (700 kDa) proteasome such as N-acetyl-L-leucinyl-L-leucinal-L-norleucinal (LLnL), N-acetyl-L-leucinyl-L-leucinyl-methional (LLM), N-carbobenzoxyl-L-leucinyl-L-leucinyl-L-norvalinal (MG115), MG132 (MyoGenics, Inc., Cambridge, Mass.), MG101, and lactacystin.

Unless otherwise defined, all technical and scientific terms used herein have the same meaning as commonly understood by one of ordinary skill in the field of molecular biology. It is noted that the term “E2A” as used herein refers to a transcription factor, as discussed above, while the term “E2” is a name historically given to a family of ubiquitin-conjugating enzymes which are distinct from transcription factor E2A, and until the present discoveries were made were believed to have no relationship to the latter. UBCE2A is a newly-discovered member of the E2 family of enzymes.

All publications, patents, and other references cited herein are incorporated by reference in their entirety.

The preferred methods, materials, and examples that will now be described are illustrative only and are not intended to be limiting. Other features and advantages of the invention will be apparent from the following detailed description, from the drawings, and from the claims.

### BRIEF DESCRIPTION OF THE DRAWINGS

FIG. 1 is a line graph depicting the degradation of the transcription factor E12 in the following pulse-chase experiment: COS cells expressing human E12 were labeled with [³⁵S] methionine for 1 hour and then chased with unlabeled methionine for 0, 60, 120, or 300 minutes. Clarified cell lysates (3×10⁵ cpm each) were subjected to immunoprecipitation with an anti-E12 antibody and analyzed by SDS-PAGE fluorography. The graph was obtained by PhosphorImaging analysis of the bands that appeared upon staining with the anti-E12 antibody and reflects the half-life of E12. [³⁵S]methionine-labeled, in vitro translated E12 migrated to the same position on the gel as the bands that were generated by staining clarified lysates from transfected COS cells with anti-E12 antibody, confirming that the latter bands were indeed E12. No signal was obtained by staining clarified lysate from COS cells that were transfected with the vector only. Similarly, immunoprecipitation of E12-transfected cells with preimmune serum gave no signal. Identical results were obtained using NIH 3T3 cells.

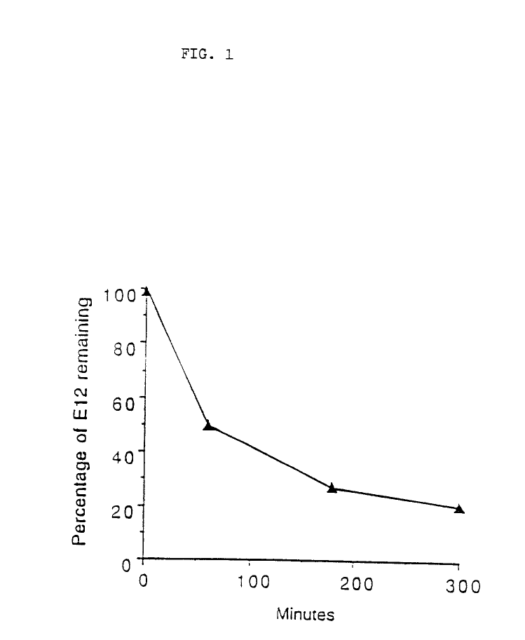

FIG. 2 is a bar graph representing the relative expression of E12 after treatment with the proteasome inhibitor MG132 and the protease inhibitor leupeptin, as follows. COS cells were electroporated with a human E12 expression plasmid. After electroporation (48 hours), cells were treated with either DMSO (a diluent for MG132), 50 μM MG132, or 1 μg/ml leupeptin for 1 hour. The cells were then pulse-chased with [³⁵S] methionine and the cell extracts were immunoprecipitated with anti-E12 antibody and analyzed by SDS-PAGE fluorography. Inhibitors were present throughout the entire pulse-chase period. The bar graph shows the quantitation of the E12 bands by PhosphorImaging analysis.

FIG. 3. is the deduced protein sequence of UBCE2A compared with that of Saccharomyces cerevisiae UBC9. The signature sequence for the ubiquitin-conjugating enzyme active site is shown in italics and the catalytic cysteine is underlined. The UBCE2A sequence contains two potential casein kinase II phosphorylation sites at positions 51 and 95 (S/T-X-X-D/E); one potential protein kinase C site at position 108 (S/T-X-R/K); and one potential cAMP/cGMP-dependent protein kinase phosphorylation site at position 48 (R/K-X-X-S/T).

FIG. 4 is a bar graph depicting the specificity of UBCE2A interactions in yeast using a quantitative β-galactosidase assay. Cells of the S. cerevisiae strain EGY48/pSH18-34 were sequentially transformed with the indicated LexA-fusion plasmid (Bait) and the AD-UBCE2A library isolate. At least three independent colonies from each AD-UBCE2A/LexA-fusion protein pair were used to inoculate a galactose-containing liquid culture. Levels of β-gal expressed from the lacZ reporter gene (normalized units) were measured; error bars indicate standard deviations.

FIG. 5A is a schematic representation of the regions of the E47 protein used as baits in the yeast two-hybrid interaction trap screen. The basic domain of E47 is shaded in black and the helix-loop-helix domain is depicted by a stippled box. The asterisk above the E47B(ALA) mutant map shows the location of the five amino acid substitutions in the basic domain. In each case, a minimum of six independent transformants were tested for galactose-inducible blue color in the presence of X-gal. The extent of color development of individual colony streaks was scored visually, with +++ indicating dark blue, +/− indicates the presence of faint blue flecks in some of the colonies and—indicating the growth of white colonies only.

FIG. 5B is a bar graph of β-galactosidase activity in yeast expressing the indicated protein pairs in the yeast two-hybrid interaction trap screen. The bar graph depicts the average values of β-galactosidase levels from experiments that were performed in duplicate on three independent isolates.

FIG. 5C is a schematic representation of the regions of UBCE2A used as interactants in the yeast two-hybrid interaction trap screen. The stippled box indicates the conserved catalytic domain of UBCE2A. Full-length human UBCH5, which was used as a control, is also depicted. In each case, a minimum of six independent transformants were tested for galactose-inducible blue color in the presence of X-gal. The extent of color development of individual colony streaks was scored visually, with +++ indicating dark blue, +/− indicates the presence of faint blue flecks in some of the colonies and—indicating the growth of white colonies only.

FIG. 5D is a bar graph depicting β-galactosidase activity using the UBCE2A constructs shown in the yeast two-hybrid interaction trap screen. The bar graph depicts the average values of β-galactosidase levels from experiments that were performed in duplicate on three independent isolates.

FIG. 6A is a line graph depicting the expression of UBCE2A MRNA during the transition from quiescence to the S phase of the cell cycle in NIH 3T3 cells. Total RNA was extracted from quiescent NIH 3T3 cells at 0, 2, 4, 7, 14, 20, and 23 hours after addition of serum. Samples of RNA (15 μg) were subjected to Northern blot analysis with random-primed DNA probes from UBCE2A and histone H3. Hybridization to an 18S rDNA probe was used to account for the variation in RNA loading. The relative intensity of each band was measured by PhosphorImaging analysis.

FIG. 6B is a line graph depicting the degree of synchronization of NIH 3T3 cells that were stimulated with serum and transitioned from quiescence to the S phase of the cell cycle in culture. The level of DNA synthesis was monitored by examining [³H] thymidine incorporation. These cells were cultured in parallel with those that were used to quantitate UBCE2A mRNA during the transition from quiesence to the S phase of the cell cycle.

FIG. 7 is a line graph depicting the inhibition of E12 degradation in cells that were transfected with antisense UBCE2A. The cells examined were from stable cell lines that were established by transfection with either vector (pCR3) or antisense UBCE2A expression plasmid (Antisense clone 3 and clone 6). These cells were transiently transfected with a human E12 expression plasmid and pulse-chase analysis was performed as described for FIG. 1. The results shown here are from one representative experiment.

FIG. 8 is a cDNA sequence encoding rat UBCE2A.

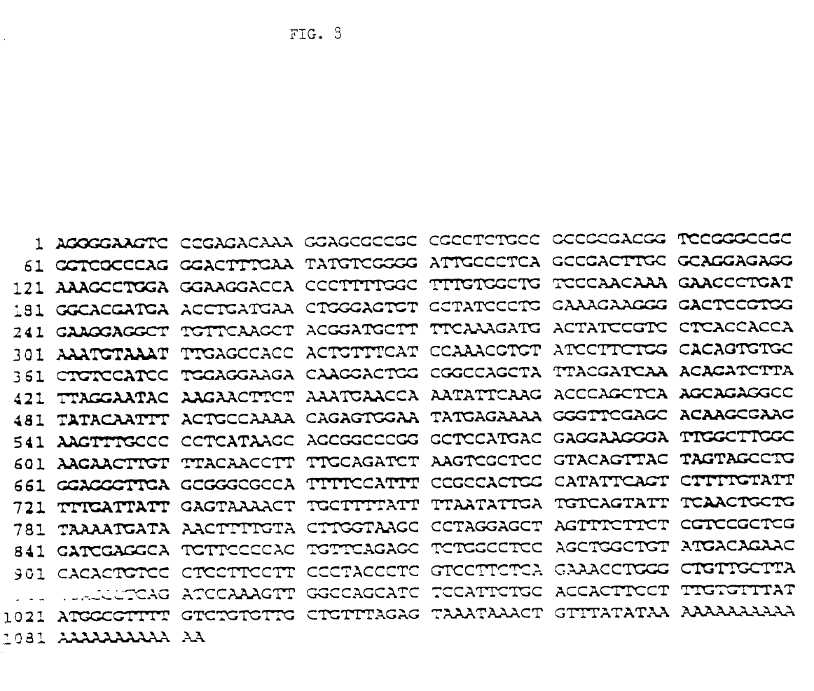

FIG. 9 is a representation of a UCBE2A antisense molecule (having the sequence of antisense clone 3 and antisense clone 6, as described herein (SEQ ID NO:6)).

### DETAILED DESCRIPTION

Experimental Reagents and Procedures

The following experimental procedures were performed in the course of the studies described herein.

Plasmids

Standard manipulations of Escherichia coli and nucleic acids were performed as described (Ausubel et al., 1993, Current Protocols in Molecular Biology, New York: John Wiley and Sons; Sambrook et al., 1989, Molecular Cloning: A Laboratory Manual, Second Edition, Cold Spring Harbor, N.Y.: Cold Spring Harbor Laboratory Press).

The following cDNAs utilized in this study have previously been described and were obtained as gifts: E12 and E47 were described by Kamps et al. (1990, Cell 60:547-555); deletion and point mutants of E47 were generated by PCR as described by Peverali et al. (1994, EMBO J. 13:4291-4301); mouse c-myc was described by Stanton et al. (1984, Nature 310:423-425); and mouse histone H3 was described by Taylor et al. (1986, J. Mol. Evol. 23:242-249).

The following cDNAs, which have also been described, were cloned by RT-PCR and confirmed by DNA sequencing: rat Id3 (Christy et al., 1991, Proc. Natl. Acad. Sci. USA 88:1815-1819); rat max (Blackwood et al., 1991, Science 251:1211-1217); human Oct 1 (Sturm et al., 1988, Genes & Dev. 2:1582-1599); and rat c-jun (Bohmann et al., Science 238:1386-1392). The ubiquitin construct, pCMVHA-Ubi, was described and donated by Treier et al. (1994, Cell 78:797-798).

For expression in eukaryotic cells, the vector pCR3 (Invitrogen) containing the CMV enhancer and promoter, and a bovine growth hormone polyadenylation signal was used. Full-length E12, UBCE2A, or c-jun cDNA was amplified by PCR and ligated into pCR3 by TA cloning (Mead et al., 1991, Biotechnology 9:657-663). The integrity of the cDNA was confirmed by dideoxy sequencing and in vitro translation of the appropriate protein.

The various deletion mutants of E12, E47, and UBCE2A were generated by standard PCR using appropriate primers followed by sequencing. CMV-HA-UBCE2A contains the sequence MASYPYDVPDYASPEF (SEQ ID NO.:4) added to the N-terminus of full-length UBCE2A. The pGEX4T vector (Pharmacia) was used for the expression of GST fusion proteins in E. coli (Smith et al., 1988, Gene 67:31-40).

Cell Culture and Antibodies

All cells were maintained in Dulbecco's modified Eagle's medium (DMEM) supplemented with 10% FCS (Hyclone), 100 U/ml penicillin, and 100 mg/ml streptomycin in a humidified atmosphere at 37° C. with 5% CO₂.

Mouse monoclonal antibody 12CA5 (Berkeley Antibody Company), anti-human E12/E47 monoclonal antibody (Pharmingen), anti-human E12 rabbit polyclonal antibody (Santa Cruz Biotechnology), anti-mouse c-jun antibody (Santa Cruz Biotechnology), goat anti-mouse IgG-HRP (Amersham), and rhodamine-conjugated anti-mouse IgG (Kirkegaard & Perry Laboratories) were used in this study. Normal rabbit and mouse sera were purchased from ICN Biochemicals.

Transfection and Immunofluorescence

NIH 3T3 cells were transfected by the calcium phosphate method (Wigler et al., 1979, Cell 14:725-731). Cells were plated at 4×10⁵ per 100 mm culture dish 16-20 hours before transfection. Fifteen micrograms of plasmid DNA was utilized for each 100 mm dish. All plasmid DNAs were prepared using a commercial DNA preparation kit (5 prime to 3 prime, Inc.), followed by purification by banding in a CsCl density gradient. Cells were transfected by the DNA-calcium phosphate method, with precipitate left in the culture medium for 22-24 hours. Following transfection, the cells were washed twice, and fed again. For transient transfections, the cells were collected by trypsinization after 24 hours, pooled, and reseeded onto 100 mm dishes. For the isolation of stable clones, the cells were split 1:10 in G418 (400 μg/ml Geneticin, Gibco) selective medium 48 hours later. The medium was changed every 3-4 days. After 18-21 days, colonies were picked using cloning cylinders and expanded. Southern blot analysis was performed to confirm integration of transfected DNA in the transformants.

Transient transfection of COS7 cells was performed by electroporation. Briefly, 5×10⁶ cells were harvested at 80% confluence and suspended in 0.8 ml phosphate-buffered saline (PBS). The cells were transferred to electroporation cuvettes (0.4 mm, Bio-Rad), mixed with 30 μg of plasmid DNA, electroporated by use of the Bio-Rad Gene Pulser at 250V and 960 mF, and then placed immediately into five 100 mm dishes.

For immunofluorescence, cells were grown to 75% confluence on chamber slides (Nunc). Cells were washed once with PBS and fixed for 20 minutes in 2% sucrose with 4% paraformaldehyde at room temperature. Fixed and permeabilized cells were hydrated in PBS for 5 minutes and incubated with 10% nonimmune rabbit serum in PBS with 0.1% Triton X-100 at room temperature for 20 minutes to suppress nonspecific binding of IgG. The slides were stained with 12CA5 (1:400 dilution) in a moist chamber for 1 hour at room temperature. After three washes in PBS with 0.1% Triton X-100, the slides were incubated with 250 μl of rhodamine-conjugated goat anti-mouse IgG diluted 1:200 for 45 minutes at room temperature. The slides were washed again extensively and counterstained with Hoechst 33258 for 5 minutes, mounted and analyzed with a Nikon fluorescent microscope. The 12CA5 staining and Hoechst staining were visualized and photographed for the same fields by changing filter sets.

Pulse-Chase Experiments and Immunoprecipitation

Cells in 100 mm dishes (either transfected cells 48 hours after transfection or stable cell lines at about 80% confluence) were starved in Met-free DMEM (supplemented with 5% dialyzed fetal bovine serum) for 60 minutes at 37° C. Cells were then pulse-labeled at 37° C. with 100 μCi/ml [³⁵S]met for 60 minutes at 37° C. Cells were chased in warm DMEM supplemented with 100 μg/ml Met. For the proteasome inhibitor experiment, the inhibitor MG132 (25 mM) was added 1 hour before pulse-chase and was present throughout the entire period. After the appropriate length of chase, dishes were washed three times with PBS, then lysed with 3 ml of ice-cold RIPA (PBS, 1% NP40, 0.5% sodium deoxycholate, 0.1% SDS, protease inhibitor cocktail [Boehringer Mannheim]) for 20 minutes at 4° C. The lysates were then cleared of nuclei and debris by centrifugation at 14,000×g at 4° C. for 15 minutes. The samples were cleared for 1 hour at 4° C. with normal mouse serum and Protein G-agarose (Pierce). Incorporation of ³⁵S into the total protein pool was determined by trichloroacetic acid (TCA) precipitation. Lysate volumes for immunoprecipitation were normalized by TCA-precipitable counts/minute. Immunoprecipitation of E12 was performed by incubating the lysates overnight at 4° C. with 1-2 μg of purified antibody and immobilized protein-G. The beads were washed four times with RIPA. SDS-PAGE was followed by fluorography. The bands were measured using a PhosphorImager (Molecular Dynamics).

Yeast Two-hybrid Interaction Trap Screeninq

The yeast two-hybrid interaction trap screening was performed according to Finley and Brent (1995, Gene Probes: A Practical Approach, Oxford University Press). EGY48 (MATa trp1 ura3 his3 LEU2::pLexop6-LEU2) was used as the host yeast strain for all interaction experiments. All bait plasmids were constructed by inserting the cDNA of corresponding genes in-frame downstream of the LexA gene contained in pEG202 (Zervos et al., 1993, Cell 72:223-232; Gyuris et al., 1993, Cell 75:791-803).

The oligo(dT)-primed rat aorta cDNA library used in the screening was constructed using the yeast galactose-inducible expression plasmid, pJG4-5 (Gyuris et al., supra). This library contains 4.5×10⁶ individual members, 88% of which contain a cDNA insert the average size of which ranges between 0.6 kb and 2.3 kb. The interaction screen was begun with a EGY48-p1840-pLexA-E12477-654 (amino acids 477 to 654 of human E12) strain. pLexA-E12477-654 gave no spontaneous transcriptional activation of either reporter used in this system. Expression of the appropriate bait protein was also confirmed by Western blot analysis using the LexA antibody or the anti-E12/E47 antibody. The library was introduced into this strain according to a variation of the procedure of Gietz et al. (1992, Nucl. Acids Res. 20:1425). A total of 4×10⁶ transformants were obtained. Screening and recovery of plasmids were performed as described by Gyuris et al. (1993, supra). Library plasmids were classified by restriction pattern after digestion with EcoRI and XhoI and either HinfI or HaeIII. Plasmid DNAs from each class were retested in the interaction-trap assay using pEG202 and pLexA-E12477-654. Galactose-inducible expression of a HA-tagged fusion protein in the transformant was also confirmed using the 12CA5 antibody.

In order to assess the specificity of interaction and to map the interaction domains, cells of the yeast strain EGY48/pSH18-34 were transformed with the indicated bait constructs and library/interactant plasmids, and plated on Ura⁻ His⁻ Trp⁻ glucose plates. The bait constructs used in the specificity test were: LexA-Id3, which contains all of the Id3 coding sequence; LexA-c-Myc, which contains the C-terminal 137 amino acids of mouse c-Myc; LexA-Max, which contains all of the rat Max coding sequences; and LexA-Oct1, which includes amino acids 294-429 of human Oct 1 (containing the POU domain). Eight to twelve colonies from each bait/interactant combination were picked and plated in duplicate on Ura⁻ His⁻ Trp⁻ X-gal plates containing either 2% glucose or 2% galactose, 1% raffinose, and the color was assessed after 48 hours.

Yeast β-gal assays of crude extracts were carried out as described by Kaiser et al. (1994, Methods in Yeast Genetics, Cold Spring Harbor Press, Cold Spring Harbor, N.Y.). Cells bearing the appropriate bait and interaction plasmids were grown to saturation overnight at 30° C. in minimal Ura⁻ His⁻ Trp⁻ medium with 2% glucose. The next day, cells were diluted 1:50 into medium containing 2% galactose and 1% raffinose and allowed to grow overnight. Lysates were then prepared and permeabilized as described by Guarente (1983, Methods Enzymol. 101:181-191). For quantitation using o-nitrophenyl-β-D-galactoside (ONPG), standard conditions were used (Guarente, supra) . Cell concentrations were determined by measuring the absorbance at 600 nm. β-gal units were calculated by the equation:

1000·(OD at 420 nm)/(time[min]·vol [ml]·OD at 600 nm).

Values reported are the average of duplicate assays of three independent transformants.

In Vitro Binding Assays

Glutathione S-transferase fusion protein expression and purification were essentially as described by Smith and Johnson (1988, Gene 67:31-40). Fresh overnight cultures of E. coli (HB101) transformed with either pGEX-4T or pGEX-4T E12477-654 were diluted 1:10 in LB medium containing ampicillin (100 mg/ml) and incubated for 3-5 hours at 37° C. with shaking until OD₆₀₀ reached 0.8. Isopropyl-β-D-thiogalactopyranoside (IPTG) was then added to a final concentration of 0.4 mM and incubation was continued for another 3 hours. Bacterial cultures were pelleted and resuspended in PBS plus 1 mM PMSF and 1% (v/v) aprotinin. The bacteria were then lysed by mild sonication at 0° C. (i.e., on ice). Triton X-100 was then added to a final concentration of 1% and the mixture was centrifuged at 14,000×g for 5 minutes at 4° C. Aliquots (1 ml) of bacterial supernatant were rocked for 30 minutes at 4° C. with 25 ml of glutathione-Sepharose 4B (Pharmacia) and the beads were then washed three times with PBS. ³⁵S-labeled proteins were generated with the TNT T7 Coupled Reticulocyte Lysate System (Promega) and the expression constructs in pCite4 (Novagen). Three ml of the ³⁵S-labeled proteins were incubated with 25 ml of beads with 50 mM NaCl and bovine serum albumin (1 mg/ml) at 4° C. for 1 hour (Shrivastara et al., 1993, Science 262:1889-1892). The beads were then washed four times with 0.1% NP-40 in PBS. Proteins on the beads were fractionated by SDS-PAGE, stained with Coomassie blue and exposed to Kodak X-ray film.

In Vivo Ubiquitination Assay

COS cells were electroporated with 6 μg of the E12 or c-jun expression construct plus 20 μg of the HA-tagged ubiquitin expression vector. After 48 hours, cells were lysed on ice in RIPA buffer plus 10 mM N-ethylmaleimide (NEM). After harvesting, cysteine was added to a final concentration of 0.1% to inactivate NEM. Immunoprecipitation was carried out as above; proteins were separated on 10% SDS-PAGE and blotted onto Immobilon-P™ membrane (Millipore). The blot was immunostained successively with 12CA5 antibody and with anti-E12 antibody. Reactive products were visualized with a peroxidase-enhanced chemiluminescent detection system (ECL; Amersham).

Yeast Complementation

YW0102 (MATa, ubc9-D1::TRP1, LEU::ubc9-1) and the wild-type strain YW01 (MATa) were utilized in this study. Yeasts were propagated on synthetic complete (SC) medium with appropriate selective omissions as described by Sherman et al. (1986, Methods in Yeast Genetics, Cold Spring Harbor Press, Cold Spring Harbor, N.Y.). The UBCE2A and UBC9 coding fragments were amplified by PCR and cloned into the plasmid, pYes2 (Invitrogen), which contains the GAL1 promoter. Lithium acetate transformation of yeast was performed by the method of Gietz et al. (1992, Nucl. Acids Res. 20:1425). Yeast transformants were plated on glucose-containing medium; colonies were picked and streaked onto galactose-containing media, and grown to colonies at 23° C. They were then streaked again onto the appropriate medium containing galactose to assay for viability at 37° C. Yeast total RNA was prepared as described in Kaiser et al. (1994, Methods in Yeast Genetics, Cold Spring Harbor Press, Cold Spring Harbor, N.Y.).

RNA Isolation and Northern Blot Analysis

Quiescent NIH 3T3 cells were serum-stimulated as described previously by Greenberg and Ziff (1984, Nature 311:433-438). [³H]thymidine incorporation was measured in triplicate from 24-well plates as described by Bowen-Pope and Ross (1982, J. Biol. Chem. 257:5161-5171). Total RNA was extracted by the RNAzolB procedure (TelTest). The rat multiple tissue mRNA blot was purchased from Clontech. For Northern analysis, total RNA (15 μg) from each time point was run on 1.2% agarose-formaldehyde gels, transferred to nitrocellulose membranes (NitroPlus™, Micron Separations), cross-linked by ultra-violet radiation and baking, and hybridized, using QuikHyb™ (Stratagene) according to the manufacturer's instructions, to the following ³²P-labeled DNA probes: an 873 bp EcoRI-XhoI fragment from the yeast interactant plasmid corresponding to full-length UBCE2A; a 1200 bp EcoRI-HindIII genomic fragment containing the entire coding sequence of mouse histone H3.2 from pH3.614, and a 18S rRNA oligonucleotide probe (ACGGTATCTGATCGTCTTCGAACC; SEQ ID NO.:3). The blots were hybridized at 55° C. and then washed twice with 2×SSC (a standardized solution of sodium chloride and sodium citrate) and 0.1% SDS (sodium dodecyl-sulfate) at room temperature for 15 minutes, followed by a 30 minute wash at 55° C. with 0.1×SSC and 0.1% SDS. Hybridization signals from the first two probes were measured and normalized to 18S rRNA.

Characterization of the Interaction between the Transcription Factor E2A and the Ubiquitin-Proteasome Pathway

The E12 Protein Is Unstable

In order to determine whether the level of E12 changes during cell cycle progression, the steady state level of the E12 protein was examined in human fibroblasts (the Hs 68 cell line) that had been made quiescent and subsequently stimulated with high serum. The cells were arrested by serum deprivation for 72 hours and reactivated with medium containing 20% serum. Total cell extracts were prepared 0, 3, 6, 9, and 12 hours after the addition of serum, and an equivalent amount of protein (70 μg) from each time point was separated by 10% SDS-PAGE. The protein was then transferred to an Immobilon-P™ filter and probed with a rabbit polyclonal antibody directed against amino acids 208-649 of E12. The bands were visualized with a horseradish peroxidase conjugated goat anti-rabbit IgG and a peroxidase-enhanced chemiluminescent detection system (ECL). E12 translated in vitro served as a positive control and staining with Coomassie Blue verified that an equivalent amount of protein was loaded in each lane.

The E12 protein level was downregulated and became barely detectable at 9 hours after serum stimulation. This result suggests that E12 is unstable and is rapidly downregulated when cells are stimulated to proliferate, providing an inverse relationship between cell growth and levels of E12 protein.

One of the features of rapidly degraded proteins is the presence of PEST sequences, which are stretches of polypeptide chain rich in proline, glutamate/aspartate, serine and threonine (Rogers et al., 1986, Science 234:264-268; Rechsteiner, 1990, Seminars Cell Biol. 1:433-440). Using the PEST-FIND program (Rogers et al., supra), three PEST regions were identified in E12: amino acids 47-67, 169-189 and 521-537). This suggests that E12 is likely to be metabolically labile.

To further explore the stability of E12, E12 turnover was studied by pulse-chase analysis. NIH 3T3 fibroblasts or COS7 cells expressing full-length E12 cDNA were pulse-labeled with [³⁵S]methionine for 60 minutes, then chased with unlabeled methionine for various times. The radiolabeled cells were lysed with ice-cold RIPA as described above, and E12 was immunoprecipitated from the clarified lysates using an anti-E12 antibody. The immunoprecipitates were analyzed by SDS-PAGE and quantified by PhosphorImager analysis. The endogenous level of the mouse homologue of E12 is low in fibroblasts (Aronheim et al., 1993, Nucl. Acids Res. 21:1601-1606; Vierra et al., 1994, Mol. Endocrinol. 8:197-209) and is not readily detectable (without endogenous labeling) using the anti-human E12 antibody utilized in these experiments. To verify that the labeled immunoprecipitated band was indeed mouse E12, the putative E12 protein and an E12 protein that was obtained from an in vitro translation system, using a rabbit reticulocyte lysate, were placed in adjacent lanes of an SDS-polyacrylamide gel and subjected to electrophoresis. The E12 protein that was obtained from the in vitro translation system had an approximate molecular weight of 72 kDa and migrated at the same position as the putative E12 protein. The experiments described above provide evidence that E12 is unstable in vivo and is degraded with an approximate half-life of 60 minutes. This short half-life may be the reason that the endogenous E12 activity in fibroblasts is low even though the mRNA is easily detectable (Metz et al., 1991, Oncogene 6:2165-2178; Watada et al., 1995, Gene 153:255-259). These observations strongly suggest that the E12 transcription factor is a target of an intracellular degradative pathway.

E12 Is Degraded Through the Ubiguitin Pathway

To investigate the proteolytic pathway that is involved in the degradation of E12, the effect of proteasome inhibitors on E12 stability was examined. A number of peptide-aldehydes, including MG101, MG115 and MG132, have been shown to be potent inhibitors of the chymotryptic site on the 20S proteasome (Rock et al., 1994, Cell 78:761-771). These inhibitors can block the degradation of long- and short-lived proteins in intact cells, as well as the proteolytic processing of antigenic peptides presented on MHC class I molecules (Rock et al., supra). In addition, MG101 and MG132 have also been shown to inhibit the degradation of the p27 inhibitor of cyclin-dependent kinases (Pagano et al., 1995, Science 269:682-686) and to block the processing of the NF-kB precursor protein p105 (Palombella et al., 1994, Cell 78:773-785).

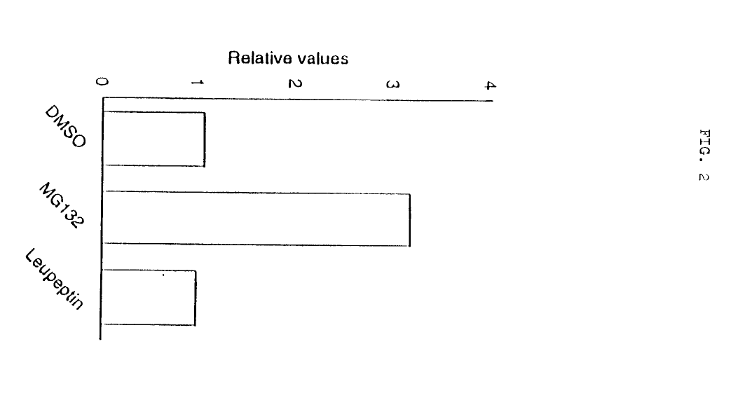

Monkey COS7 cells were transfected with a human E12 expression plasmid. Forty-eight hours after transfection, the cells were treated with the proteasome inhibitors MG132 or lactacystin for 1 hour. Dimethyl sulfoxide (DMSO) or the protease inhibitor, leupeptin, were used as controls. The cells were then pulse-labeled with [³⁵S]methionine for 60 minutes, followed by a 3 hour chase period with unlabeled methionine. Cell lysates were immunoprecipitated with anti-E12 antibody, and the proteins were separated by SDS-PAGE. E12 protein was stabilized in the presence of MG132 whereas DMSO or leupeptin (1 μg/ml) had no effect (FIG. 2). Therefore, the degradation of E12 involves the proteasome.

Since degradation of a protein via the proteasome involves tagging of the protein by covalent attachment of multiple ubiquitin molecules (Ciechanover, 1994, Cell 79:13-21; Jentsch et al., 1995, Cell 82:881-884), the ubiquitination assay developed by Treier et al. (1994, Cell 78:787-798) was utilized to determine whether E12 can be ubiquitinated in vivo. In these experiments, the E12 expression plasmid together with a hemagglutinin (HA)-tagged ubiquitin expression vector were introduced into COS7 cells by transient transfection. c-Jun, which is multiubiquitinated, was used as a control (Treier et al., supra). Cell lysates were prepared in the presence of N-ethylmaleimide, which inactivates many enzymes of the ubiquitin pathway, including activities of the ubiquitin-dependent protease and the ubiquitin hydrolases (Goebl et al., 1994, Mol. Cell. Biol. 14:3022-3029), and equivalent amounts were subjected to immunoprecipitation using either an E12 or a c-Jun antibody. The precipitated proteins were separated by SDS-PAGE, blotted onto Immobilon-P™ membranes, and probed with a monoclonal anti-HA antibody (12CA5). A horseradish peroxidase-enhanced chemiluminescent detection system (ECL) was used to visualize bound antibodies. With c-Jun-transfected cells, a faint ladder of bands that exceeded Mr 39,000, which is the relative molecular mass of c-Jun, was seen, with the bulk of the reactivity at Mr>200,000. This indicates the formation of multiple ubiquitin conjugates. A similar observation was made for E12-transfected lysates. In fact, the ladder of bands appeared to be even more distinct than with c-Jun-transfected lysates. Again, the appearance of high molecular mass conjugates indicates significant ubiquitination. Expression of E12 in these cells was confirmed by reacting the same blot with an anti-E12 antibody. In both cases, no bands were recognized in vector-transfected cells. The demonstration that E12 is ubiquitinated and that its degradation can be inhibited by proteasome inhibitors strongly suggests that the ubiquitin-proteasome pathway plays a role in regulating the abundance of this transcription factor.

Novel Ubiquitin-Conjugating Enzyme Cloned by the Interaction Trap System

To identify proteins that interact with the C-terminus of E12 the yeast interaction trap cloning system (Gyuris et al., 1993, Cell 75:791-803) was employed. A bait expression vector was constructed by fusing the LexA-binding domain to the C-terminus of E12 (amino acids 477-654), which includes the bHLH domain. This construct (LexA-E12477-654) gave no basal transcriptional activity to either of the reporter genes (LEU2 and LacZ) used in this system.

A rat aorta cDNA expression library was screened and 42 positive clones out of 3.5×10⁶ transformants were identified. All of the potentially positive clones demonstrated galactose-dependent growth in medium lacking leucine and turned blue on 5-bromo-4-chloro-3-indolyl β-D-galactoside plates. Of these clones, 29 encoded Id3 (Christy et al., 1991, Proc. Natl. Acad. Sci. USA 88:1815-1819) and 5 encoded Id1 (Benezra et al., 1990, Science 251:1211-1217). This demonstrates that specific protein-protein interactions are detectable using the E12 construct, as described herein, in yeast. The remaining clones were assigned to four different classes, one of which encodes a novel ubiquitin-conjugating enzyme based on the presence of the highly conserved enzyme active site. This gene was named UBCE2A.

Ubiquitin-conjugating enzyme (also referred to as E2) selectively catalyzes the covalent attachment of ubiquitin to proteins targeted for degradation. Therefore, E2 plays an important role in the ubiquitin-proteasome proteolytic pathway (Jentsch, 1992, Ann. Rev. Genet. 26:179-207). The identification of UBCE2A as a protein that interacts with E12 would suggest that UBCE2A plays a regulatory role in the turnover of the transcription factor E12.

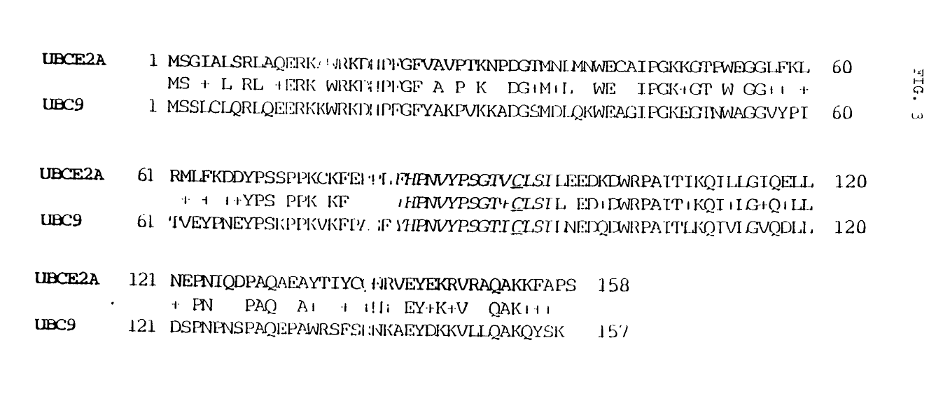

Sequence comparison of the predicted amino acid sequence of UBCE2A to all known E2 sequences revealed that it is most homologous to Saccharomyces cerevisiae UBC9 (56% identity, 75% similarity; Seufert et al., 1995, Nature 373:78-81) and Schizosaccharomyces pombe hus5 (66% identity, 82% similarity; Al-Khodairy et al., 1995, J. Cell Sci. 108:475-486; see also FIG. 3 herein). In budding yeast, UBC9 is an essential nuclear ubiquitin-conjugating enzyme that is involved in the degradation of S- and M-phase cyclins (Seufert et al., supra). In pombe, hus5 mutants are severely impaired in growth and exhibit high levels of abortive mitoses (Al-Khodairy et al., supra). Therefore, it is likely that UBCE2A belongs to the family of E2 enzymes that may function in many aspects of cell cycle progression.

The UBCE2A Protein

To examine the subcellular localization of UBCE2A protein, COS7 cells were transfected with a plasmid that expressed the protein linked with the HA epitope. The cells were analyzed by indirect immunofluorescence, as follows. The cells were fixed and stained with a monoclonal anti-HA antibody, 12CA5, and the antigen-antibody complex was detected with secondary antibodies that were fluorescently-tagged with rhodamine or fluorescein isothiocyanate (FITC). Counterstaining with Hoechst 33258 showed that the UBCE2A protein was primarily expressed in the nucleus. No staining was seen when COS cells were transfected with the same vector lacking insert. Furthermore, immunoblot analysis of nuclear extracts prepared from UBCE2A-transfected cells revealed an approximately 18 kDa protein, which is consistent with the expected molecular mass of UBCE2A. This result demonstrates that the UBCE2A protein localizes to the nucleus, and thus is in a position to act on E2A nuclear factors.

To demonstrate that UBCE2A has ubiquitin conjugation activity and that it may be a homologue of UBC9, a growth complementation experiment was performed in yeast. We made use of a ubc9 temperature-sensitive (ts) mutant (ubc9-1) in which growth is arrested when the cells are incubated at 37° C. (Seufert et al., 1995, Nature 373:78-81). Full-length UBCE2A and UBC9 were cloned into pYes2, a 2 micron plasmid (InVitrogen) that directs expression from the galactose-inducible GAL1 promoter. These constructs were introduced into ubc9-1, and galactose-dependent growth at 37° C. was assayed. The UBC9 transformants readily rescued the ts phenotype, whereas no growth was seen with the vector transformants. UBCE2A transformants were also able to rescue the ts phenotype, although their growth was slower than that of the UBC9 transformants. The lesser effectiveness of UBCE2A in growth complementation may reflect species-specific structural differences between UBCE2A and UBC9. Alternatively, it may mean that UBCE2A may be a member of a different UBC family than UBC9.

Specific Interactions In Vitro

The interaction trap provides a reliable qualitative measure of protein-protein interactions (Estojak et al., 1995, Mol. Cell. Biol. 15:5820-5829). Therefore, this method was used to further evaluate the specificity of the interaction between E12 and UBPCE2A.

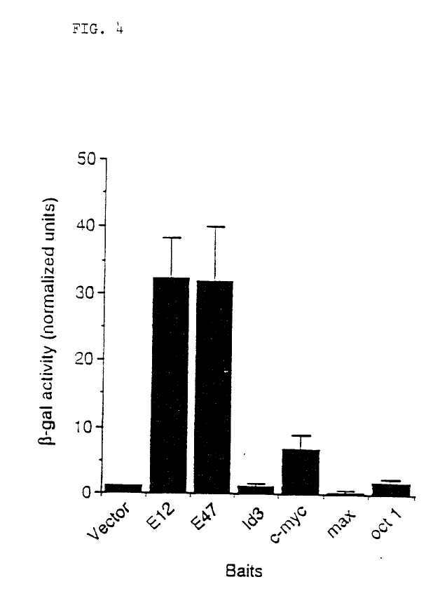

Full-length UBCE2A fused to the B42 transcription activation domain (AD-UBCE2A) was introduced into yeast cells containing different LexA fusion proteins, and transcriptional activity was measured using β-galactosidase assays. Lysates from yeast bearing LexA-E12477-654 or LexA-E47477-651 and AD-UBCE2A contained about 20-fold more β-gal activity than a strain bearing AD-UBCE2A and LexA (FIG. 4). This result also indicates that both E12 and E47 interact equally well with UBCE2A and that the primary amino acid sequence within the differentially spliced region is not crucial for binding.

The specificity of the interaction partners was further examined by transforming yeast harboring expression plasmids encoding LexA fusions with various known HLH proteins. No interaction was detected with the HLH protein, Id3 (Christy et al., 1991, Proc. Natl. Acad. Sci. USA 88:1815-1819), the bHLH-leucine zipper protein, max (Blackwood et al., 1991, Science 251:1211-1217), or the homeodomain protein, Oct 1 (Sturm et al., 1988, Genes & Dev. 2:1582-1599); only weak promoter activity was discerned following introduction of LexA-myc. LexA-myc has also been shown to result in higher background LacZ expression when used with other proteins (Cuomo et al., 1994, Proc. Natl. Acad. Sci. USA 91:6156-6160). Western blot analysis was used to confirm the expression of the appropriate LexA fusion proteins.

To confirm the interaction observed in yeast, radiolabeled in vitro translated UBCE2A was precipitated with glutathione S-transferase (GST) E12 immobilized on glutathione-Sepharose beads. As predicted, UBCE2A associates with GST-E121-654 or GST-E12477-654 but not with GST. [³⁵S]methionine-labeled in vitro translated UBCE2A was also immunoprecipitated in the presence of in vitro translated E12 protein using an anti-E12 antibody. Therefore, there is a specific interaction between E12 and UBCE2A.

Mapping of Interacting Regions

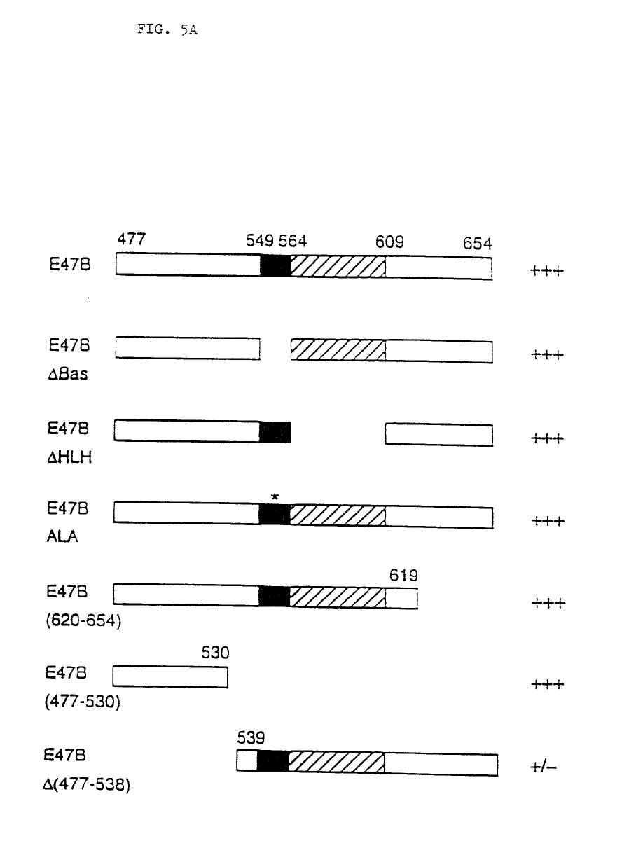

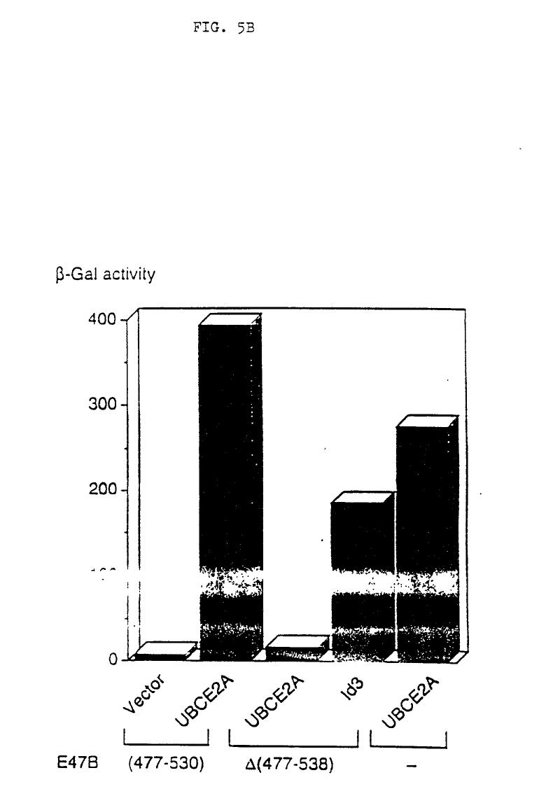

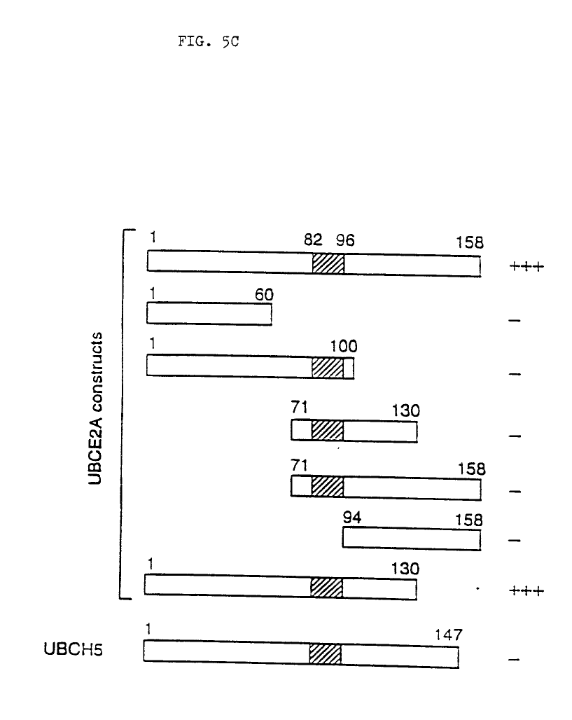

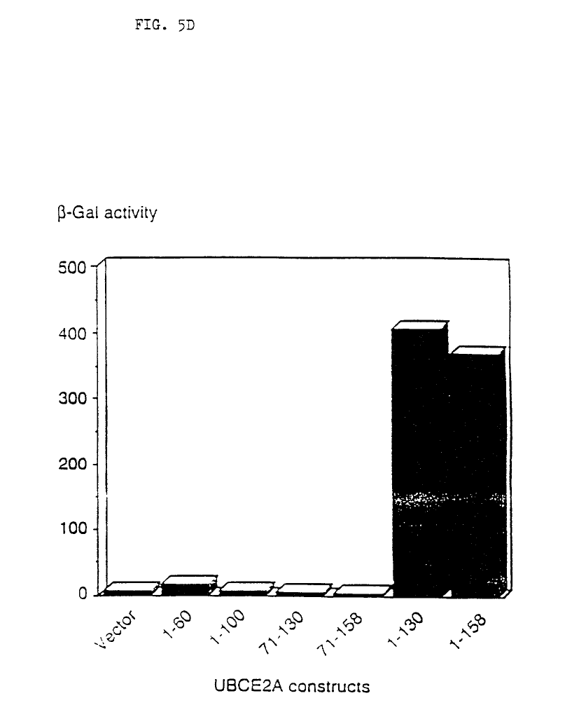

To determine if the bHLH domain of E12 mediates binding to UBCE2A, a number of deletion mutants were generated in the bHLH domain and assayed for transcriptional activity using the interaction trap system. Both E12 and E47 are fully capable of interacting with UBCE2A, a non-HLH protein, suggesting that the bHLH domain may not be important in this case, although it is involved in the dimerization with other HLH proteins like myoD and Id (Murre et al., 1989, Cell 56:777-783; Benezra et al., 1990, Cell 61:46-59). As predicted, deletions of either the basic or the HLH region have no effect on UBCE2A binding to E47 (FIG. 5A and FIG. 5B). Similarly, mutations in the basic domain that affect DNA-binding and transactivation activities (Chakraborty et al., 1991, J. Biol. Chem. 266:2827-2882) did not abrogate binding. More extensive mapping localizes the binding site to a 54-amino acid region of E47, amino acids 477-530 (FIG. 5A and FIG. 5B). This region is conserved in both E12 and E47. This region by itself can confer specific binding to UBCE2A. Moreover, LexA-E12 lacking this region (LexA-E12539-654) binds to Id3 but has no affinity for UBCE2A. One characteristic of this region is that there is a high local concentration of lysine residues that could serve as potential sites for ubiquitination (Chau et al., 1989, Science 243:1576-1583). This result defines a novel interaction domain in E12 that may play a role in regulating its turnover.

The binding site in UBCE2A was also defined. All of the clones that were recovered from the interaction trap encoded full-length protein, suggesting that either the N-terminus or the entire protein is required for interaction. Sequential deletions were made in both the N- and C-termini of UBCE2A and the resulting polypeptides were tested for binding to E12 residues 477-654, identified above. Similar results were obtained using E12 residues 477-530. The findings indicate that almost the entire UBCE2A protein, including the conserved catalytic site, is required for binding; only about 29 amino acids at the C-terminus are dispensable (FIG. 5C and FIG. 5D). One explanation for the failure to detect an interaction between LexA-E12477-530 and the deletion mutants of AD-UBCE2A could have been that the AD fusion proteins were poorly expressed. To address this possibility, a portion of each lysate used to measure β-galactosidase activity was subjected to gel electrophoresis and blot transfer, followed by detection with anti-HA antiserum (12CA5). AD-fusion proteins of the appropriate size were detected in each of the lysates, making it unlikely that failure to detect interaction in vivo could be attributed to degradation or inadequate synthesis of the chimeras. The specificity of this interaction was confirmed by demonstrating that neither E12477-530 nor E12477-654 binds to UBCH5 (Scheffner et al., 1994, Proc. Natl. Acad. Sci. USA 91:8797-8801), a human E2 enzyme that is involved in the ubiquitination of p53 (FIG. 5C and FIG. 5D). This study suggests that a particular conformation of UBCE2A is required for interaction. Alternatively, specific complex formation between UBCE2A and another cellular protein is necessary for targeting the enzyme to E12.

Expression of UBCE2A mRNA

E2A mRNA has been found in all tissues examined, and its presence in E-box binding complexes suggests a broad expression pattern (Murre et al., 1989, Cell 58:537-544; Roberts et al., 1993, Proc. Natl. Acad. Sci. USA 90:7583-7587). To investigate the expression pattern of UBCE2A, Northern blot analysis was performed on poly(A)-selected RNA from multiple rat tissues. Two transcripts, of 2.1 and 1.1 kb, were detected in all tissues examined, with the exception of testis where a third transcript of 1.5 kb was also seen. Lung showed the lowest level of expression. The 1.1 kb transcript is relatively more abundant except in brain where the larger transcript is predominant. The rat UBCE2A cDNA obtained in the screen described above is ˜1 kb and most likely represents the lower transcript. The 2.1 kb transcript may be a product of a related gene or an alternatively spliced form of the UBCE2A gene.

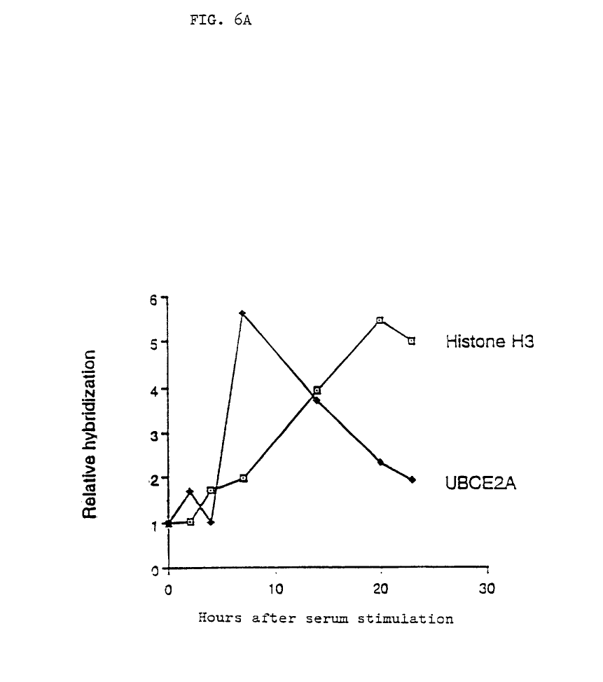

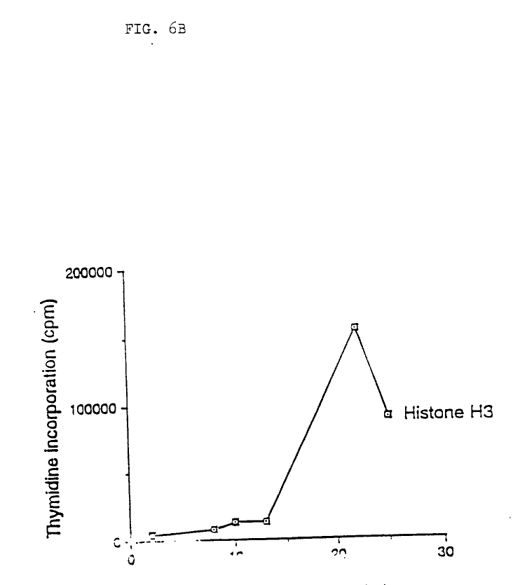

The expression of UBCE2A during the cell cycle was also examined. RNA was isolated from quiescent NIH 3T3 cells, and from NIH 3T3 cells that were stimulated to proliferate by the addition of serum to the medium (FIG. 6A). The degree of cell synchrony was monitored by the level of DNA synthesis and the presence of histone H3 mRNA, an S phase-expressed gene (FIG. 6B). Northern analysis indicates that the expression of UBCE2A MRNA peaks during G1 phase and begins to drop in early S phase. A similar pattern of expression has been observed in rat vascular smooth muscle cell cultures. This timing of expression would suggest that the UBCE2A enzyme could function during late Gl phase to inhibit the growth arrest mediated by E2A proteins.

In addition, it has been determined that the level of UBCE2A expression increases two-fold in the rat carotid artery within three days of balloon injury, as occurs frequently in the course of angioplasty. This observation strengthens the conclusion that UBCE2A-mediated degradation of E12/E47 plays an important role in regulating the response of vascular smooth muscle cells to injury.

Overexpression of Antisense UBCE2A mRNA Stabilizes E12

One of the major functions of a ubiquitin conjugating enzyme is to catalyze the transfer of an activated ubiquitin moiety to a specific lysine residue of a target protein. This conjugation reaction may require accessory proteins known as ubiquitin ligases (or E3s) for substrate recognition (Ciechanover, 1994, Cell 79:13-21). Following formation of a conjugate between ubiquitin and the target protein, the protein moiety of the adduct is degraded by the proteasome (Jentsch et al., 1995, Cell 82:881-884).

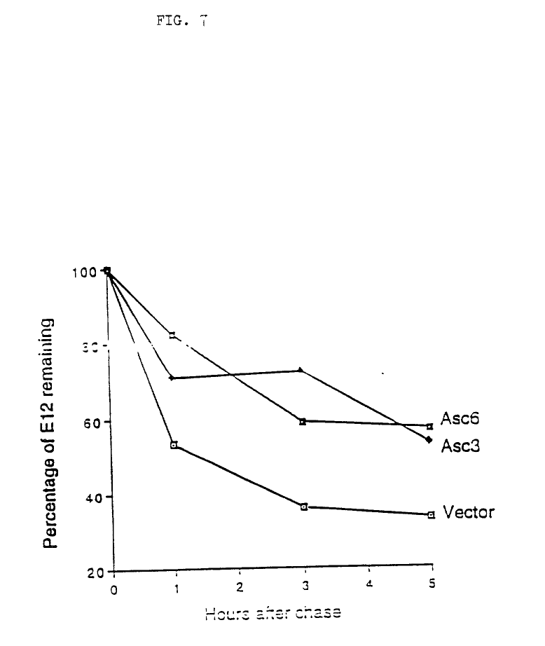

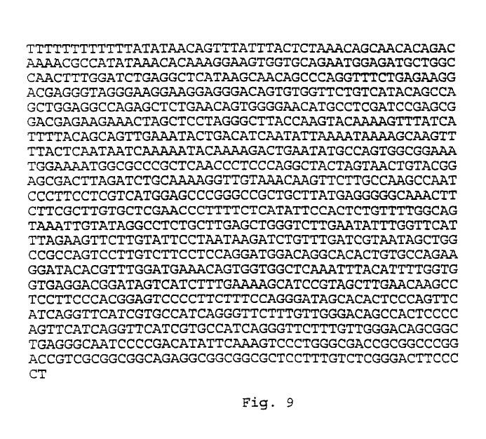

To investigate the specific role of UBCE2A in the degradation of E12, an antisense UBCE2A cDNA sequence was introduced into NIH 3T3 cells by transfection. Two antisense clones, Asc3 and Asc6, and a vector-transfected clone were studied. The sequence of Asc3/Asc6 is shown in FIG. 9 (SEQ ID NO:6). Decreased levels of the 1.1 kb UBCE2A mRNA were seen in Asc3 and Asc6 cells: the level of UBCE2A mRNA in Asc3 and Asc6 cells was about 30% and 32%, respectively, of the UBCE2A mRNA level in vector control cells, as measured by Northern blot analysis using an antisense riboprobe, ³²P-labeled UBCE2A. These cells were then transiently transfected with an E12 expression plasmid and pulse-chase analysis as described above was performed 48 hours later. The results indicated that in both antisense clones, the E12 protein was stabilized when compared to vector clone (FIG. 7) or the parental cell. The initial rate of degradation was reduced and an approximate 2-fold stabilization of E12 was observed. It is apparent that the UBCE2A enzyme plays an important role in regulating the level of E12 protein in the cell.

Since the E2A proteins are involved in tissue-specific gene transcription, converting cells from all or nearly all tissue types from a proliferative to a differentiated state, UBCE2A may be an attractive therapeutic target for regulating cellular differentiation mechanisms. Examples of the methods whereby UBCE2A may be targeted are presented below.

The discovery of UBCE2A and its role in the degradation of the transcription factor E2A could benefit a human patient who is suffering from any unwanted proliferative growth of cells. This proliferative growth could be associated with a malignant or benign tumor, a leukemia, a lymphoma, or a vascular injury, including vascular injuries that result from surgeries such as balloon angioplasty. There are at least four ways to inhibit cellular proliferation by reducing the UBCE2A-mediated degradation of E2A. These include treatment with: (1) proteasome inhibitors, (2) anti-UBCE2A antibodies, (3) UBCE2A antisense oligonucleotides, and (4) mutant E2A proteins that lack a UBCE2A binding site or lack the lysine residues which are targets for ubiquitination.

Treatment with Proteasome Inhibitors

The particle responsible for the major neutral proteolytic activity in the cell is the proteasome, a 20S (700 kDa) particle that functions as the proteolytic core of a large complex that degrades ubiquitin-conjugated proteins (Rock et al., 1994, Cell 78:761-771; Orlowski, 1990, Biochem. 29:10289-10297; Rivett, Biochem. J. 291:1-10).

Compounds that inhibit the proteasome and that are suitable for in vivo application have recently been discovered. The compounds are peptide-aldehydes and include N-acetyl-L-leucinyl-L-leucinal-L-norleucinal (LLnL), N-carbobenzoxyl-L-leucinyl-L-leucinyl-L-norvalinal (MG115), and N-acetyl-L-leucinyl-L-leucinyl-methional (LLM). Rock et al. (supra) demonstrated that these proteasome inhibitors were not toxic to either T or B lymphoblastoid cells: protein synthesis was unaffected, and the cells remained intact and excluded vital dyes. Furthermore, the peptide-aldehydes readily penetrated the cell membrane, and rapidly and effectively inhibited proteolysis. Thus, peptide aldehydes are potentially suitable for clinical application.

Compounds inhibit the proteosome could be administered to a patient singly or in combination, through a variety of routes that are well known to persons skilled in the art of pharmacology. A preferred route is topical application, which could be accomplished at the same time as a related surgical procedure. For example, a therapeutic composition containing peptide aldehydes could be placed in the area where a tumor had been removed. Similarly, such a therapeutic composition could be applied through the catheter used to perform an angioplasty, or could be coated on the balloon itself.

If required, there are numerous ways to facilitate the delivery of peptide aldehydes. For example, they could be packaged within a liposome. The liposome would be created by dissolving the peptide aldehyde in an aqueous solution, adding appropriate phospholipids and lipids, possibly with surfactants, and dialyzing or sonicating the mixture.

Peptide aldehydes that inhibit the proteasome can also be incorporated into microspheres, which are composed of well known polymers. The advantage associated with microspheres is that they can be implanted for slow release over a period of time, or tailored for passage from the gastrointestinal tract into the bloodstream. The slow release of peptide aldehydes can also be achieved in a local area by incorporating them into a pluronic solution that forms a gel at normal body temperature. Detailed methods regarding liposomes, microspheres, and pluronic solutions can be found in the following publications: U.S. Pat. Nos. 4,789,734, 4,925,673, and 3,625,214, the review by Gregoriadis in Drug Carriers in Biology and Medicine (1979, Academic press, p. 287-341), and Simons et al. (1992, Nature 359:67-70).

The dosage and length of any treatment are known to depend on the nature of the disease or injury and to vary from patient to patient as a function of age, weight, sex, and general health, as well as the particular compound to be administered, the time and route of administration, and other drugs being administered concurrently. Skilled artisans will be guided in their determination of peptide-aldehyde dosages by the studies of Rock et al. (supra), who examined the proteolysis of ovalbumin after application of peptide-aldehydes and found that these compounds differed in their efficacy: MG115 was approximately 5-fold more potent than LLnL and caused a 50% inhibition of ovalbumin degradation at 0.4 μM. In contrast, LLM did not affect ovalbumin degradation at concentrations up to 100 μM.

Treatment with anti-UBCE2A Antibodies

A patient who is suffering from an undesirable proliferation of cells may also be treated with agents that specifically inhibit the activity of UBCE2A. One of the ways to inhibit UBCE2A activity is by taking advantage of the specificity of antigen-antibody interactions: antibodies that specifically bind and neutralize the activity of UBCE2A can be used to elevate cellular levels of E2A, which will, in turn, inhibit cellular proliferation.

The antibodies used in this therapeutic approach may be intact monoclonal or polyclonal antibodies, genetically engineered antibodies, humanized antibodies, or antibody fragments, including F(ab′)2, Fab′, Fab, Fv, and sFv fragments. They may be administered to the patient as polypeptides, or expressed from recombinant nucleic acids introduced into the proliferating cells. Skilled artisans will have ready access to information regarding the methods for generating such antibodies or antibody fragments, including the following publications: Ladner (U.S. Pat. Nos. 4,946,778 and 4,704,692) describes methods for preparing single polypeptide chain antibodies; Ward et al. describe the preparation of heavy chain variable domains, termed “single domain antibodies,” which have high antigen-binding affinities (1989, Nature 341:544-546); Boss et al. (U.S. Pat. No. 4,816,397) describe various methods for producing immunoglobulins and immunologically functional fragments thereof, which include at least the variable domains of the heavy and light chain in a single host cell; and Cabilly et al. (U.S. Pat. No. 4,816,567) describe methods for preparing chimeric antibodies. Monoclonal antibodies with a desired binding specificity can be commercially humanized (Scotgene, Scotland; Oxford Molecular, Palo Alto, Calif.), and fully human antibodies can be generated in transgenic animals (Green et al., 1994, Nature Genetics 7:13-21).

Anti-UBCE2A antibodies may be administered by any standard route, including intraperitoneally, intramuscularly, subcutaneously, intravenously, or topically. It is expected, however, that the preferred routes of administration will be intravenous and topical application. The topical application could be performed at the time of a related surgical procedure, such as tumor ablation or angioplasty, as described above.

The dosage of an anti-UBCE2A antibody will depend on many factors, including those reviewed above in the discussion of treatment with proteasome inhibitors. The dosages for intravenous administration are typically approximately 0.1 to 100 μg/ml blood volume, or 0.1 to 100 mg/kg body weight. Skilled artisans will be further guided in their determination of adequate dosage by previous antibody-dependent therapies. For example, Abraham et al. (1995, J. Amer. Med. Assoc. 273:934-941) administered a murine TNF-α monoclonal antibody to human patients at doses of 1 to 15 mg/kg. This therapy was well tolerated by all patients, despite the development of human anti-murine antibodies. Similarly, Rankin et al. (1995, Br. J. Rheumatol. 34:334-342) administered a single intravenous dose of 0.1, 1.0 or 10 mg/kg of an engineered human antibody, CDP571 that neutralized human TNF-α. These studies, taken together with the availability of methods to generate numerous types of highly-specific antibodies, provide a strong basis for anti-UBCE2A antibody-based therapies.

Treatment with UBCE2A Antisense Oligonucleotides

A second means of inhibiting the activity of UBCE2A is through the use of antisense UBCE2A oligonucleotides. These oligonucleotides are capable of inhibiting the expression of UBCE2A by a mechanism which is believed to involve blocking either the transcription of the UBCE2A gene or the translation of UBCE2A mRNA. The underlying mechanism is presumed to rely on hybridization interactions, but other mechanisms may also be involved.

These oligonucleotides would consist of 10 or more nucleotides linked in a sequence that is the complement of, i.e. antisense to, at least a portion of the sequence of the sense strand of a gene encoding UBCE2A, or of UBCE2A mRNA. It is expected that these oligonucleotides would be introduced into a target cell in one of two ways: either by direct introduction of the antisense oligonucleotide into the cell, or by introduction into the cell of a DNA which is transcribed within the cell to produce multiple copies of an antisense RNA. In the latter instance, the DNA sequence which is to be transcribed in the cell could be linked, by standard recombinant techniques, to transcriptional control sequences that direct expression within a cell that is in need of UBCE2A downregulation, but not in other cell types. Another means of selectively targeting cells can be achieved by linking oligonucleotides to molecules that are natural ligands to the targeted cell, or by use of a vector, such as a retrovirus, which is taken up primarily by proliferating cells. Oligonucleotides may cross the cell membrane spontaneously. In addition, their entry may be facilitated, particularly when an expression vector is used, by any standard transfection technique, such as via a liposome, as described above.

A therapeutically effective amount is an amount of the antisense molecule of the invention which is capable of producing a medically desirable result in a treated animal. A preferred dosage for intravenous administration of nucleic acid is approximately 10⁶ to 10²² copies of the nucleic acid molecule. As described above, a particularly relevant application of the current invention is the prevention of cellular proliferation following balloon angioplasty. For this purpose, skilled artisans will be especially aided by the study of Simons et al. (1992, Nature 359:67-70) wherein antisense c-myb oligonucleotides were added to pluronic solutions at 1 mg/ml and applied to a denuded portion of the carotid artery.

Where the antisense oligonucleotide itself is the therapeutic that is administered, it will probably be desirable to employ certain backbone modifications to make the oligonucleotide more resistant to enzymatic degradation. Methods for doing so are well known in the art of antisense technology. For example, the oligonucleotide can be stabilized with phosphotriester linkages, or by modifying the backbone with phosphorothioates, methylphosphonates, phosphorodithioates, phosphoroamidates, phosphate esters, or other molecules. The 3′ end of an oligonucleotide may also be linked to aminoacridine or polylysine to help protect from endonucleases.

Methods of antisense design and introduction into host cells are described, for example, in Weinberg et al., U.S. Pat. No. 4,740,463, and therapeutic applications can be found in the following review articles: Le Doan et al., 1989, Bull. Cancer 76:849-852; Dolnick, 1990, Biochem. Pharmacol. 40:671-675; Crooke, 1992, Ann. Rev. Pharmacol. Toxicol. 32:329-376; Uhlman and Peyman, 1990, Chemical Reviews 90:544-584; 1990, Anticancer Research 10:1169-1182.

Treatment with Mutant E2A Proteins

The discovery of the UBCE2A-mediated degradation of E2A suggests a fourth type of cellular anti-proliferative treatment. This treatment relies on the use of mutant E2A proteins. These mutants, which can be constructed by standard recombinant DNA techniques, would function as transcription factors but lack the UBCE2A binding site of wild-type E2A, which lies within the 54 amino acid region defined by the studies described herein. Alternatively, the E2A mutant could merely lack one or more of the lysine residues to which UBCE2A typically links a molecule of ubiquitin, rendering the mutant less likely to be “tagged” with the ubiquitin molecules which trigger proteolysis of E2A.

Administration of these mutants to human patients in the form of the polypeptide itself or an expression vector encoding the polypeptide requires consideration of the same factors as detailed in the treatment regimes described above, such as route of administration and dosage.

Identification of Additional UBCE2A Homoloques and Splice Variants

The discovery and cloning of UBCE2A allow additional UBCE2A homologues and splice variants to be readily identified in rat and other species. UBCE2A homologues or splice variants can be identified in a given species by, for example, screening a genomic or CDNA library generated from that species with an appropriate UBCE2A cDNA probe under conditions that will allow the probe to hybridize with the UBCE2A gene(s) or cDNA(s), of that species. Methods for generating and screening libraries are well known to persons skilled in the art of molecular biology. In addition, genomic and cDNA libraries from many species are commercially available.

A second standard technique that could be used is PCR-based cloning, employing PCR primers derived from the rat UBCE2A cDNA (SEQ ID NO.:1). Alternatively, one could utilize the same methodology described above for cloning rat UBCE2A CDNA. Of particular interest are the human and murine UBCE2A homologues.

Preparation of Purified UBCE2A and UBCE2A Fragments

The polypeptides of the invention may be purified from a biological sample, chemically synthesized, or produced recombinantly. For example, a suitable host cell may be transformed with all or part of an UBCE2A-encoding CDNA fragment in a suitable expression vehicle. Those skilled in the field of molecular biology will understand that any of a wide variety of expression systems may be used to produce the recombinant UBCE2A polypeptide. The precise host cell used is not critical to the invention. The UBCE2A polypeptide may be produced in a prokaryotic host (e.g., E. coli) or an a eukaryotic host (e.g., yeast, such as Saccharomyces cerevisiae; insect cells, such as Sf-9 cells; or mammalian cells, such as COS-1, NIH 3T3, and JEG3 cells). Such cells are available from a wide range of sources, e.g., the A.T.C.C. (also see Ausubel et al., supra). The method of transfection and the choice of expression vehicle will depend on the host system selected. Standard transformation and transfection methods are described, e.g., by Ausubel et al. (supra); expression vehicles may be chosen from, e.g., those described in Cloning Vectors: A Laboratory Manual (P. H. Pouwels et al., 1985, Supp. 1987) and in Ausubel et al. supra.

One example of an expression system that may be used is a mouse 3T3 fibroblast host cell transfected with a pMAMneo expression vector (Clonetech, Palo Alto, Calif.). pMAMneo provides: an RSV-LTR enhancer linked to a dexamethasone-inducible MMTV-LTR promoter, an SV40 origin of replication, which allows replication in mammalian systems, a selectable neomycin gene, and SV40 splicing and polyadenylation sites. DNA encoding an UBCE2A polypeptide can be inserted into the pMAMneo vector in an orientation designed to allow expression. The recombinant UBCE2A could then be isolated as described below. Other host cells that may be used in conjunction with pMAMneo, or similar expression systems, include COS cells and CHO cells (A.T.C.C. Accession Nos. CRL 1650 and CCL 61, respectively).

UBCE2A polypeptides may also be produced in stably-transfected mammalian cell lines. A number of vectors suitable for stable transfection of mammalian cells are available to the public, e.g., see Pouwels et al. (supra); methods for constructing such cell lines are well known in the art (see, e.g., Ausubel et al., supra). In one example, cDNA encoding UBCE2A is cloned into an expression vector which includes the dihydrofolate reductase (DHFR) gene. Integration of the plasmid and, therefore, the UBCE2A-encoding gene into the host cell chromosome is selected for by inclusion of 0.01-300 μM methotrexate in the cell culture medium (see, e.g., Ausubel et al., supra). This dominant selection can be accomplished in most cell types. Recombinant protein expression can be increased by DHFR-mediated amplification of the transfected gene. Methods for selecting cell lines bearing gene amplifications are described in Ausubel et al. (supra); such methods generally involve extended culture in medium containing gradually increasing levels of methotrexate. DHFR-containing expression vectors commonly used for this purpose include pCVSEII-DHFR and pAdD26SV(A), which are described in Ausubel et al. (supra). Any of the host cells described above or a DHFR-deficient CHO cell line (e.g., CHO DHFR⁻ cells, A.T.C.C. Accession No. CRL 9096) are among the host cells that may be used for DHFR selection of a stably-transfected cell line or DHFR-mediated gene amplification. Other useful expression systems include cell-free expression systems and transgenic animals who produce the desired polypeptide in their milk; in the latter case, the UBCE2A polypeptide would probably have to be expressed fused to an appropriate secretion signal peptide.

Purification of UBCE2A Polypeptides

Once an UBCE2A polypeptide is expressed, as described above, it may be isolated using standard methods, such as affinity chromatography. For example, E2A or an antibody against UBCE2A may be attached to a column and used to isolate the UBCE2A polypeptide. Lysis and fractionation of UBCE2A-harboring cells prior to affinity chromatography may be performed by standard methods (see, e.g., Ausubel et al., supra). The recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Fragments of UBCE2A polypeptides can also be produced by chemical synthesis (e.g., by the methods described in Solid Phase Peptide Synthesis, 2nd ed., 1984, The Pierce Chemical Co., Rockford, Ill.).

Preparation of anti-UBCE2A Antibodies and UBCE2A Antibody Fragments

Purified UBCE2A polypeptides may be used to generate antibodies that specifically bind to UBCE2A. These antibodies may be prepared by a variety of standard techniques. For example, the UBCE2A polypeptide, or an antigenic fragment thereof, can be administered to an animal in order to induce the production of polyclonal antibodies. Alternatively, standard hybridoma technology can be used to prepare monoclonal antibodies. In addition, genetically engineered, neutralizing, and/or humanized antibodies that bind UBCE2A can be generated by well known methods, as can antibody fragments, including F(ab′)2, Fab′, Fab, Fv, and sFv fragments. As described above, skilled artisans have ready access to information regarding the methods for generating such antibodies or antibody fragments, including the publications of Ladner (supra), Ward et al. (supra), Boss et al. (supra), Cabilly et al. (supra), and Green et al. (supra).

Preparation and Screening of UBCE2A Mutants

Given the discovery of the activity of the UBCE2A protein, and the DNA sequence that encodes it, as well as the structure/function information provided above, skilled artisans are well equipped to identify mutants of UBCE2A which either retain or lose the ability to ubiquitinate E2A. As a first step in this process, standard techniques could be employed to introduce site-specific point mutations within a sequence encoding wild type UBCE2A. Alternatively, these sites could be mutated by deletion. With the mutant protein in hand, skilled artisans could perform the ubiquitination assay developed by Treier et al. (1994, Cell 78:787-798), or the yeast complementation assay, to determine which mutant UBCE2A proteins retained the ability to ubiquitinate E2A, and which mutants failed to retain this activity. The above disclosure provides substantial guidance as to what mutants might be expected to be active and which inactive. For example, it is expected that any of the 29 carboxy-terminal residues of wild type UBCE2A can be deleted or altered without affecting the E2A-binding and ubiquitinating activity of the resulting mutant polypeptide.

Other embodiments are within the scope of the claims.

DEPOSIT

Under the terms of the Budapest Treaty on the International Recognition of the Deposit of Microorganisms for the Purpose of Patent Procedure, deposit of a plasmid bearing a UBCE2A cDNA sequence has been made with the American Type Culture Collection (A.T.C.C.) of Rockville, Md., USA, where the deposit was given Accession Number 97492.

Applicants' assignee, President and Fellows of Harvard College, represent that the A.T.C.C. is a depository affording permanence of the deposit and ready accessibility thereto by the public if a patent is granted. All restrictions on the availability to the public of the material so deposited will be irrevocably removed upon the granting of a patent. The material will be available during the pendency of the patent application to one determined by the Commissioner to be entitled thereto under 37 C.F.R. 1.14 and 35 U.S.C. §122. The deposited material will be maintained with all the care necessary to keep it viable and uncontaminated for a period of at least five years after the most recent request for the furnishing of a sample of the deposited plasmid, and in any case, for a period of at least thirty (30) years after the date of deposit or for the enforceable life of the patent, whichever period is longer. Applicants' assignee acknowledges its duty to replace the deposit should the depository be unable to furnish a sample when requested due to the condition of the deposit.

1. A substantially pure polypeptide that (1) catalyzes the covalent attachment of ubiquitin to E2A, and (2) has a sequence that is at least 70% identical to SEQ ID NO.:2.

2. The polypeptide of claim 1, wherein said polypeptide has the sequence of a mammalian UBCE2A.

3. The polypeptide of claim 1, wherein the sequence of said polypeptide comprises SEQ ID NO.:2.

4. An isolated DNA molecule encoding the polypeptide of claim 1.

5. The DNA molecule of claim 4, wherein said polypeptide has the sequence of a mammalian UBCE2A.

6. The DNA molecule of claim 4, wherein the sequence of said polypeptide comprises SEQ ID NO.:2.

7. The DNA molecule of claim 5, wherein said DNA molecule hybridizes to a probe consisting of a sequence that is complementary to the coding sequence of SEQ ID NO.:1 when hybridized and washed under the following stringency conditions: 55° C., 0.1×SSC, 0.1% SDS.

8. A vector comprising the DNA molecule of claim 4.

9. A cell comprising the DNA molecule of claim 4.

10. A method of making a polypeptide, said method comprising:
(a) culturing the cell of claim 9 under conditions permitting expression of said polypeptide from said DNA, and
(b) harvesting said polypeptide from said cell or from the medium surrounding said cell.

11. An antibody that specifically binds a mammalian UBCE2A.

12. A method of inhibiting the proliferation of a cell, said method comprising:
(a) identifying an animal having a cell the proliferation of which is susceptible to being inhibited by increasing the level of the transcription factor E2A in the cell; and
(b) introducing into the cell a proteasome inhibitor.

13. A method of inhibiting the proliferation of a cell, said method comprising:
(a) identifying an animal having a cell the proliferation of which is susceptible to being inhibited by increasing the level of the transcription factor E2A in the cell; and
(b) introducing into the cell a mutant E2A that possesses the transcription factor activity of wild type E2A but that lacks (i) the UBCE2A binding site of wild-type E2A, or (ii) at least one of the lysine residues which are ubiquitination sites on wild-type E2A.

14. A method of inhibiting the proliferation of a cell that expresses the transcription factor E2A, said method comprising introducing into the cell a compound that reduces the level of UBCE2A biological activity in the cell.

15. The method of claim 14, wherein said compound is an anti-UBCE2A antibody.

16. The method of claim 14, wherein said compound is a single-stranded nucleic acid at least 12 nucleotides in length that is antisense to at least a portion of the coding strand of said cell's naturally-occurring gene or MRNA encoding UBCE2A.

17. An isolated, single-stranded DNA molecule at least 12 nucleotides in length which is antisense to at least a portion of the coding strand of a naturally-occurring gene or MRNA encoding UBCE2A.

18. An isolated molecule of DNA that is transcribed into an MRNA that: (1) is approximately 1.1, 1.5, or 2.1 kilobases in length; and (2) hybridizes to a DNA probe consisting of a sequence that is complementary to (a) the coding sequence of SEQ ID NO.:1, or (b) a naturally occurring MRNA encoding human UBCE2A, when hybridized and washed under the following conditions: 55° C., 0.1×SSC, 0.1% SDS.

19. A substantially pure polypeptide encoded by the DNA of claim 18.

20. A substantially pure polypeptide consisting of a mutant form of the mammalian transcription factor E2A that differs from wild type E2A in that it (1) is unable to bind UBCE2A, and so cannot be ubiquitinated by UBCE2A; or (2) lacks one or more of the lysine residues that are ubiquitination sites on wild type E2A.

21. A DNA molecule comprising SEQ ID NO:6.
